(19) United States
(12) Patent Application Publication
(10) Pub. No.: US 2025/0345073 A1
(43) Pub. Date: Nov. 13, 2025

(54) GUIDE SYSTEM FOR EXTREMITIES AND RELATED METHODS

(71) Applicant: Forma Medical, Inc., Camp Hill, PA (US)

(72) Inventors: James A. Gault, Lincoln, RI (US); Andrew Charles Davison, Hummelstown, PA (US); John Randolph Clements, Roanoke, VA (US)

(73) Assignee: Forma Medical, Inc., Camp Hill, PA (US)

(21) Appl. No.: 19/203,266

(22) Filed: May 9, 2025

Related U.S. Application Data
(60) Provisional application No. 63/645,765, filed on May 10, 2024.

(51) Int. Cl. A61B 17/16; A61B 17/17; A61B 17/34; A61B 17/86; A61B 90/00

(52) U.S. Cl. CPC A61B 17/1682; A61B 17/1697; A61B 17/1732; A61B 17/3472; A61B 17/864; A61B 90/39; A61B 2090/3966

(57) ABSTRACT

A system configured to guide one or more wires or fixation elements toward one or more bone segments of extremity bones is provided.

### CROSS-REFERENCE TO RELATED APPLICATIONS

The present application claims priority to and the benefit of U.S. Provisional Application No. 63/645,765, filed May 10, 2024, the contents of which are hereby incorporated in its entirety.

### TECHNICAL FIELD

The present disclosure relates to a surgical system for extremity bones and related methods, in particular, to a guide system.

### BACKGROUND

A common procedure for handling healing of broken bones and addressing deformities such as hammertoe is the use of bone fixation implants for fusing one or more adjacent bones. Conventional bone fixation implants utilize generic screws and wires that create a rigidly fused joint with very limited adjustability intraoperatively. Some implants offer some limited degree of flexibility and/or adjustment when used under very specific circumstances that require highly technical surgical procedures. Such existing bone fixation implants often require multiple components with many intricate mating features requiring customization depending on the type of bone, patient, or desired location of the implant in the body of a patient. This results in increased costs, less desirable healing outcomes, and multiple procedures to achieve a desired outcome.

### SUMMARY

An embodiment of the present disclosure includes a surgical system. The surgical system includes a guide instrument with a guide body having a proximal end, a distal end spaced from the proximal end along longitudinal axis, an upper surface and a lower surface spaced from the upper surface along a vertical axis that is perpendicular to the longitudinal axis. The guide instrument includes a distal platform having a set of spaced-apart grouping members along the upper surface. Each grouping member having a plurality of through-holes that extend through the guide body. And each through-hole is configured to receive therethrough a separate fixation wire. The guide instrument includes a sighting member carried by the distal platform, the sighting member defining a sighting channel that extends in direction parallel to or along the longitudinal axis. The guide instrument includes a back guide member located proximal with respect to the distal platform. The back guide member has a plurality of guide barrels each with a channel that extends along a direction aligned with or along the longitudinal axis.

### BRIEF DESCRIPTION OF THE DRAWINGS

The foregoing summary, as well as the following detailed description of exemplary embodiments of the present application, are better understood when read in conjunction with the appended drawings. For the purposes of illustrating the present application, shown in the drawings are exemplary embodiments of the disclosure. It should be understood, however, that the application is not limited to the precise arrangements and instrumentalities shown. In the drawings:

FIG. 1 is a perspective view of a surgical system according to an embodiment of the present disclosure;

FIG. 2 is a perspective view of a guide instrument in the system shown in FIG. 1;

FIG. 3 is a top view of the guide instrument of the system shown in FIG. 1;

FIG. 4 is a bottom view of a guide instrument of the system shown in FIG. 1;

FIG. 5 is a side view of the guide instrument shown in FIG. 4;

FIG. 6 is a side view of a guide instrument of the system shown in FIG. 1;

FIG. 7 is a back view of a guide instrument of the system shown in FIG. 1;

FIG. 8 is a front view of the guide instrument shown in FIG. 7;

FIG. 9 is a perspective view of a trocar of the system shown in FIG. 1; and

FIG. 10 is a side view of the trocar shown in FIG. 9; and

FIGS. 11-16 illustrate a method for guiding a device toward a bone according to an embodiment of the present disclosure.

### DETAILED DESCRIPTION OF ILLUSTRATIVE EMBODIMENTS

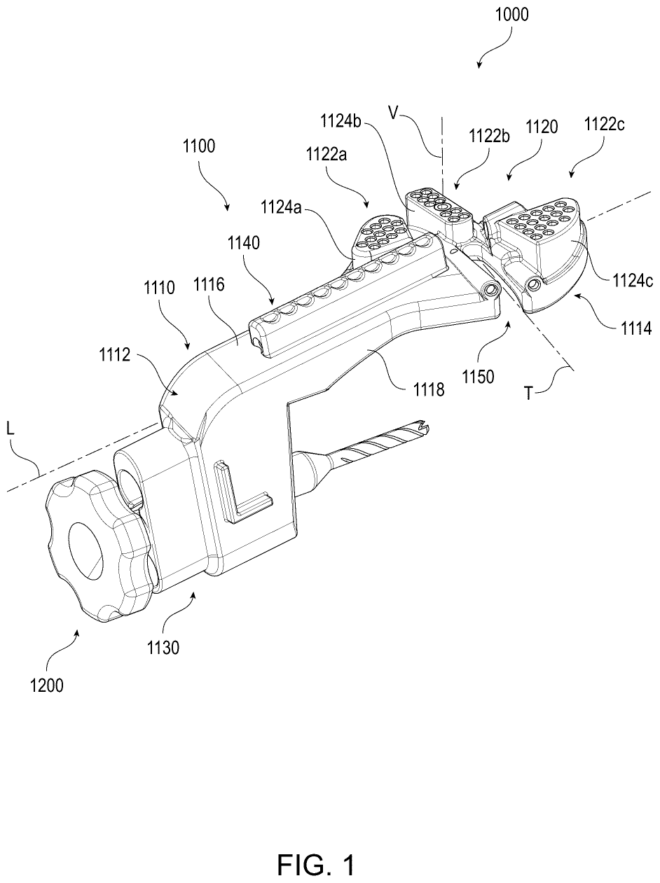

Surgical systems, guidance systems and fixation devices described in this application are configured to aid in the fixation of two or more bones or bone segments, typically in extremity bones, such as the foot. Referring to FIG. 1, a surgical or guiding system 1000 as described herein are configured for fixation of toe and/or for hammertoe correction, for example. In one example, the systems are suitable for use with the AKIN procedure which includes a medical closing osteotomy of the proximal phalanx of the large toe. In addition, the systems and methods described herein may be configured for interphalangeal joint fixation. For example, the guiding systems may be used for fixation of metatarsals, proximal phalanges, middle phalanges, or distal phalanges. While the embodiments described are configured for interphalangeal joint fixation, it is possible that the described embodiments could be configured for fixation of phalanges, metatarsals, cuneiform, or cuboid bones in the foot. In other embodiments, systems may be used for fixation of bone segments of phalanges, metatarsals, or other bones in the hand.

As shown in FIG. 1, a guiding system 1000 may be used to guide various devices, e.g. instruments and fixation elements, toward a target bone. The guiding system 1000 includes a surgical instrument 1100 and trocar 1200. Additional components, such as wires, fixation elements, drills, cannulas and the like may also be included in the system 1000.

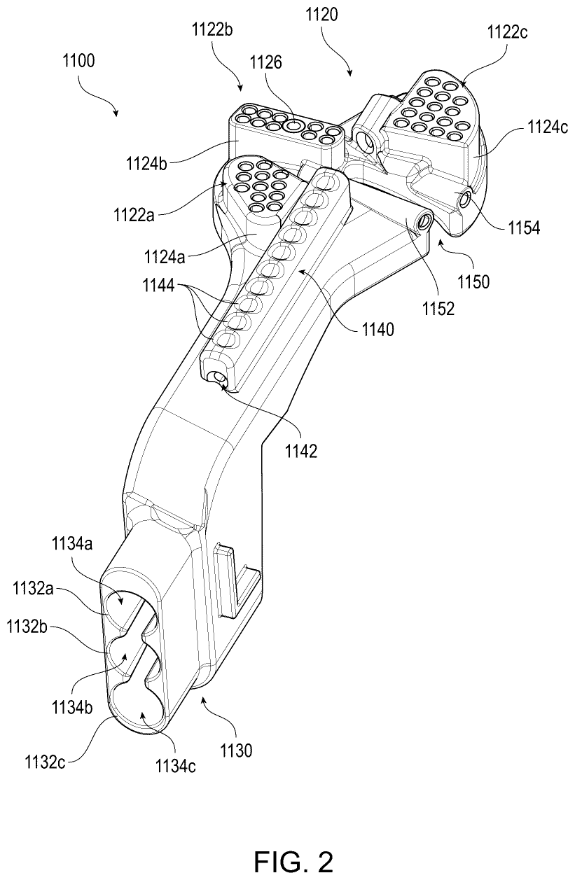

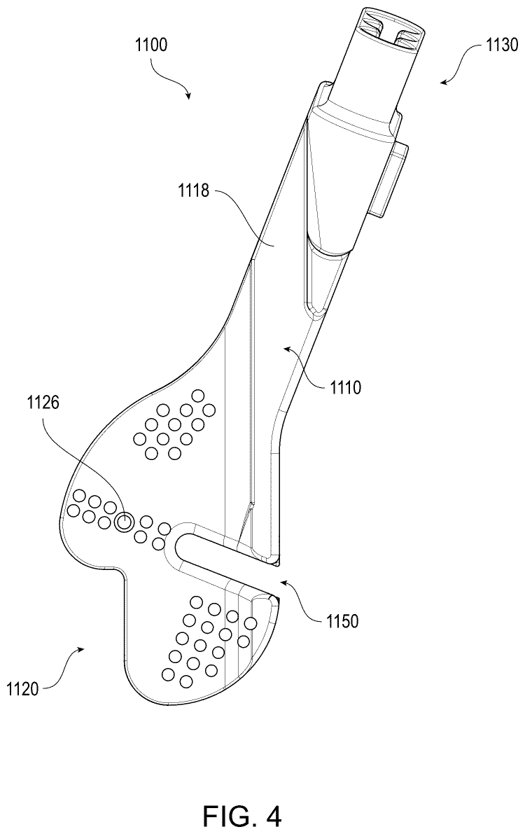

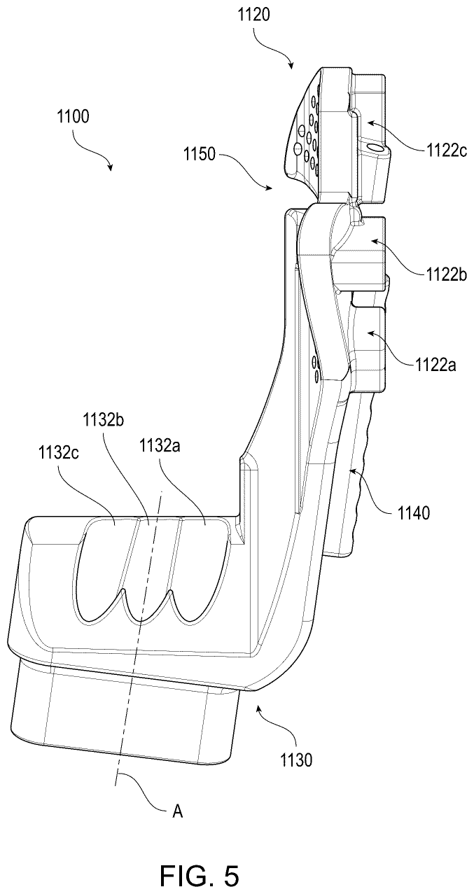

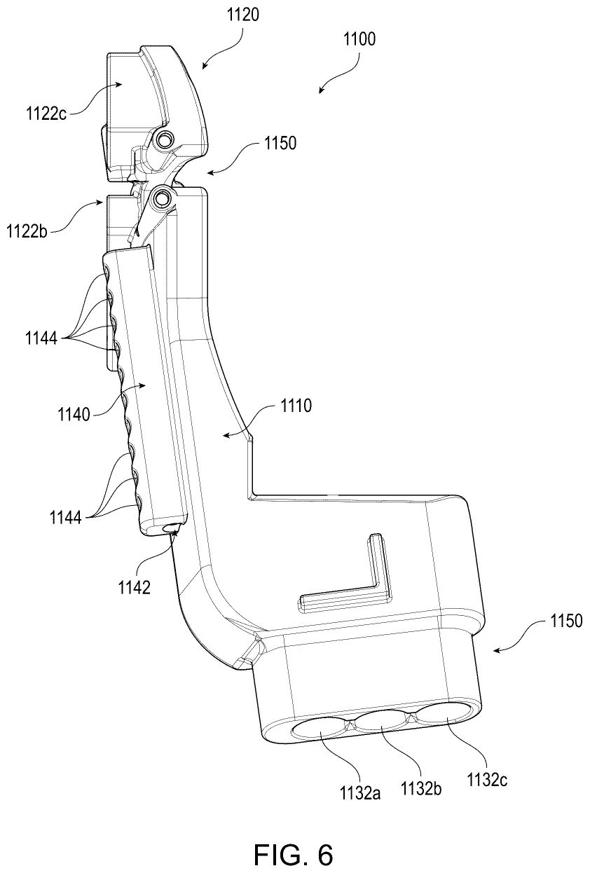

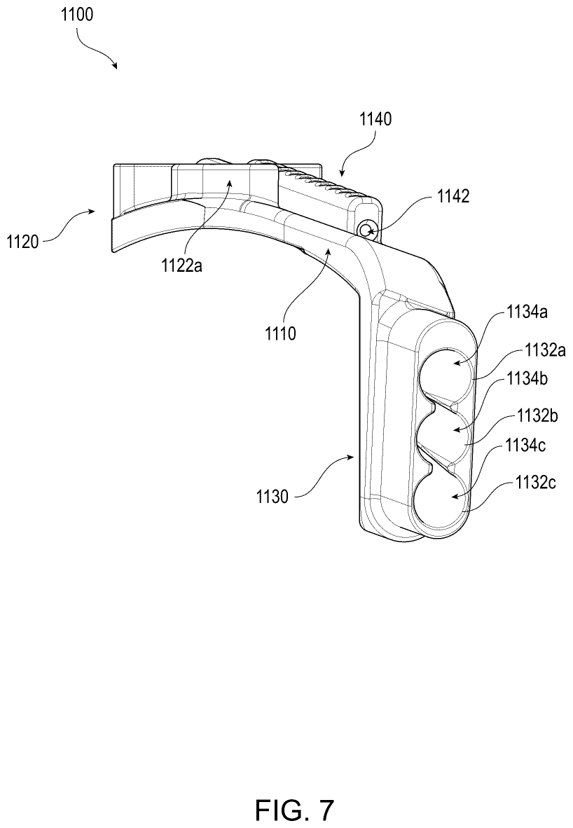

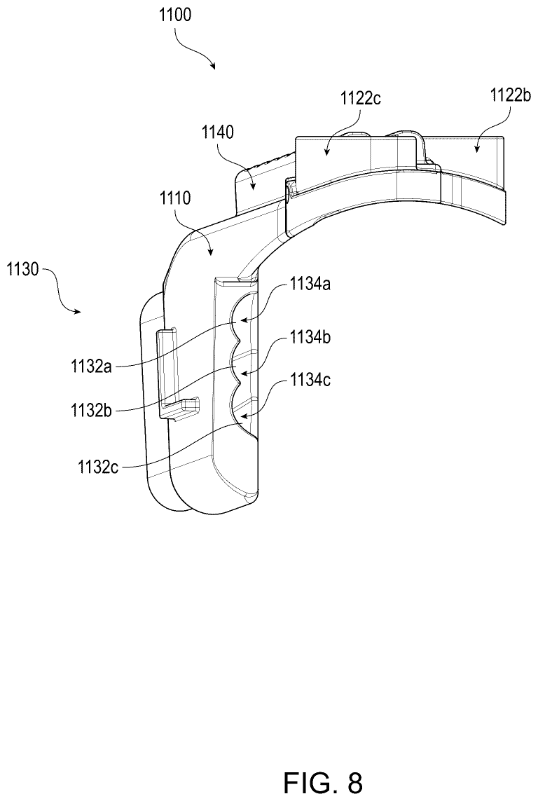

Referring to FIGS. 2-8, the surgical instrument 1100 includes a guide body 1110 having a proximal end 1112, a distal end 1114 spaced from the proximal end 1112 along longitudinal axis L, an upper surface 1116 and a lower surface 1118 configured to face tissue. The guide body 1110 also includes a distal platform 1120 and a back guide member 1130. The lower surface of the distal platform 1120 may be planar or curved to mate with the tissue or bone. In some embodiments, the lower surface of the distal platform 1120 is a curved concave surface. The distal platform 1120 has a set of spaced apart grouping members 1122a, 1122b, 1122c carried by a respective set of protrusions 1124a, 1124b, 1124c extending upwardly in a vertical direction.

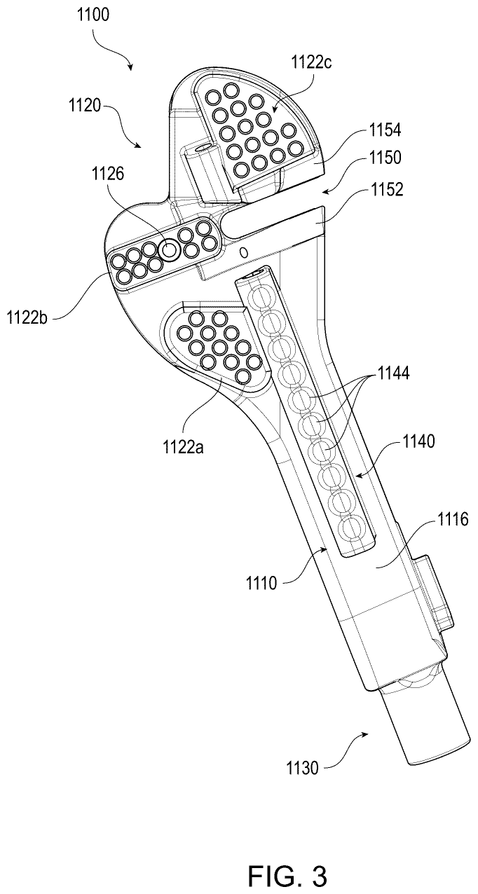

As shown in FIGS. 2 and 3, each grouping member 1122a, 1122b, 1122c includes a plurality of through-holes configured to receive therethrough a fixation wire. As shown, the first grouping member 1122a may have between two and twenty through-holes. In some embodiments, the first grouping member 1122a may have between about six and twelve through-holes. A second grouping member 1122b may have between two and twenty through-holes. In some embodiments, the second grouping member 1122b may have between about six and twelve through-holes. A third group member 1122c may have between two and twenty through-holes. In some embodiments, the second grouping member 1122b may have between about eight and eighteen through-holes. Each grouping member 1122a, 1122b, 1122c is spaced apart relative to each, both in along a longitudinal axis L and a transverse axis T. In this way, the first grouping member 1122a may be considered a proximal grouping member, the second grouping member 1122b may be considered a central grouping member (which is shown in drawings offset relative to other two grouping members) and the third grouping member 1122c may be considered the distal grouping member. The presence and spacing of the sets of grouping members 1122a, 1122b, 1122c, and their respective through-holes, allow fixation wires to be sequentially inserted through the through-holes for temporary fixation to tissue as the procedure is conducted, as will discussed in more detail below. It will be understood to one skilled in the art that each of the grouping members 1122a, 1122b, 1122c can be provided with one or more through-holes, and the total number of through-holes for each of the grouping members 1122a, 1122b, 1122c can be adjusted according to the overall size the surgical instrument 1100 and the intended anatomy size. For example, a surgical instrument intended for pediatric surgical operations may contain fewer through-holes, and correspondingly fewer fixation wire placement options, than a surgical instrument intended for adult surgical operations. During operation, the user may select a hole from each of the grouping members 1122a, 1122b, 1122c that is most appropriate for the patient's anatomy.

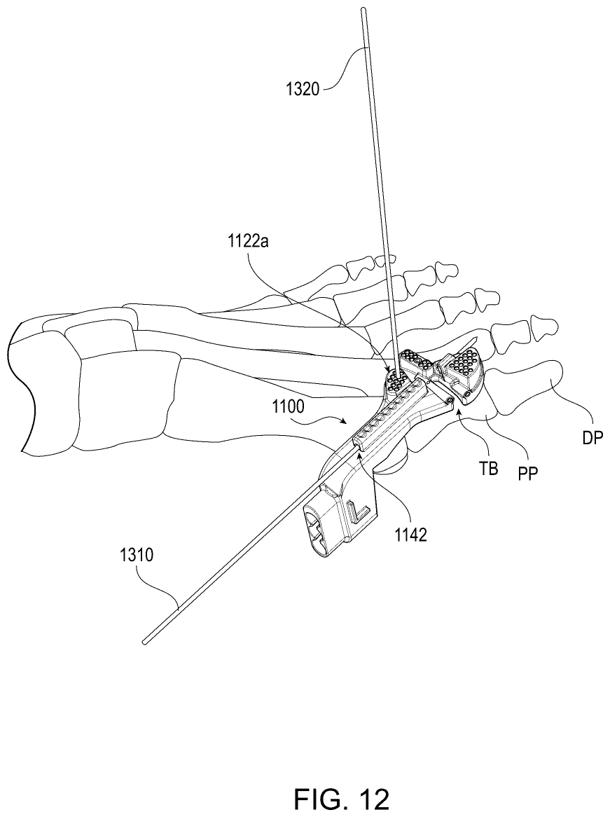

Continuing with FIGS. 2-8, the guide instrument 1100 has a sighting member carried by at least the distal platform of the guide body 1100. The sighting member 1140 defines a sighting channel 1142 that extends in direction parallel to or along the longitudinal the axis L. This sighting channel allows for a “sighting wire” 1310 to inserted therethrough (see FIG. 12). The sighting channel 1142 is configured to, when the surgical instrument 1100 is placed adjacent the tissue, to be placed above and aligned the screw k-wire holes. On top of the sighting member 1140 are indents 1144 to allow for easy clamp fixation if desired. As shown, the sighting channel 1142 enables a sighting wire 1310 to be positioned dorsal to a screw k-wire that is inserted through the back guide member 1130 via a guide channel as will be described in further detail below.

The guide instrument 1100 includes an osteotomy window 1150 for facilitating a bone osteotomy. The osteotomy window 1150 extends into the distal platform 1120 along a direction that is transverse to the longitudinal axis L and a vertical axis V. In some embodiments, the osteotomy window 1150 extends into the distal platform 1120 with a width of about 5 to 10 mm and a length of about 15 to 30 mm.

The guide body 1110 includes various radio-opaque elements to aid in visualization. For instance, the guide body 1110 may include a radio-opaque ring carried by one of the grouping members 1122a, 1122b, 1122c. In the embodiment shown, the radio-opaque ring 1126 centered along the vertical axis V that is perpendicular to a plane including the longitudinal axis L, and is positioned on the second, or central grouping member 1122b. In some embodiments, the vertical axis V is perpendicular to the longitudinal axis L (not shown). Given its position and orientation, the radiopaque ring 1126 can be used to obtain a true AP directional x-ray by obtaining a perfect circle under fluoroscope. In addition, the guide instrument 1100 includes a first elongated radio-opaque element 1152 in the guide body 1110 adjacent to the osteotomy window 1150, and a second elongated radio-opaque element 1154 in the guide body 1110 adjacent to the osteotomy window 1150 and opposite the first elongated radio-opaque element 1152. The first elongated radio-opaque elements 1152 may be elongated rods with a length that exceeds its diameter, the length being about the same length of the osteotomy window 1150.

The guide instrument 1100 includes a back guide member 1130 located proximal with respect to the distal platform 1120. The back guide member 1130 extends from the lower surface 1118 of the guide body 1110. In some embodiments, the back guide member 1130 extends from lower surface 1118 of the guide body 1110 with a height of about 40 to 80 mm. The back guide member 1130 having a plurality of guide barrels 1132a, 1132b, 1132c configured to receive a shaft therethrough. More specifically, each guide barrel 1132a, 1132b, 1132c has guide channel 1134a, 1134b, 1134c. The guide barrels 1132a, 1132b, 1132c are generally positioned in a vertical relation with respect to each other. As shown, there are three guide barrels 1132a, 1132b, 1132c having three guide channels 1134a, 1134b, 1134c, respectively. Although the back guide member 1130 is shown as having three guide barrels 1132a, 1132b, 1132c with respective guide channels 1134a, 1134b, 1134c for receiving a trocar, in some embodiments, the back guide member 1130 may include a first mounting portion for receiving a second mounting portion of an movable guide barrel (not shown). The movable guide barrel is configured to slidably or removably mount to different vertical locations of the first mounting portion such that a height of the movable guide barrel can be adjusted relative to the lower surface 1118 of the guide body 1110. In some embodiments, three guide barrels 1132a, 1132b, 1132c may be replaced with a single slot that permits a trocar to be positioned vertically at different heights and/or angled relative to the lower surface 1118 of the guide body 1110.

Additionally, in some embodiments, the guide barrels 1132a, 1132b, 1132c may be replaced with smaller slots or openings to receive and guide a screw k-wire directly without the use of a trocar. The slots or openings may further be sized for cannulated drills and cannulated screws to pass through without interference. In some embodiments, the back guide member 1130 may include a plurality of slots or openings spaced vertically from the lower surface 1118 of the guide body 1110. In some embodiments, the back guide member 1130 may include a single vertical column of one to ten slots or openings. In some embodiments, the back guide member 1130 may include two to five vertical columns, with each column having one to ten slots or openings.

In some embodiments, the guide instrument 1100 includes a front guide member (not shown) that extends downward from a lower surface of the distal platform 1120. The front guide member includes openings or markings that are axially aligned with plurality of guide barrels 1132a, 1132b, 1132c. The openings or markings may be used by the user to orient the guide instrument 1100 on the anatomy, and/or to provide the user with guidance on the trajectory of the trocar and/or screw k-wire once those are inserted into one or more of the plurality of guide barrels 1132a, 1132b, 1132c. In some embodiments, the front guide member may be made of a transparent or translucent material to enhance visualization of the openings or markings of the front guide member over the anatomy.

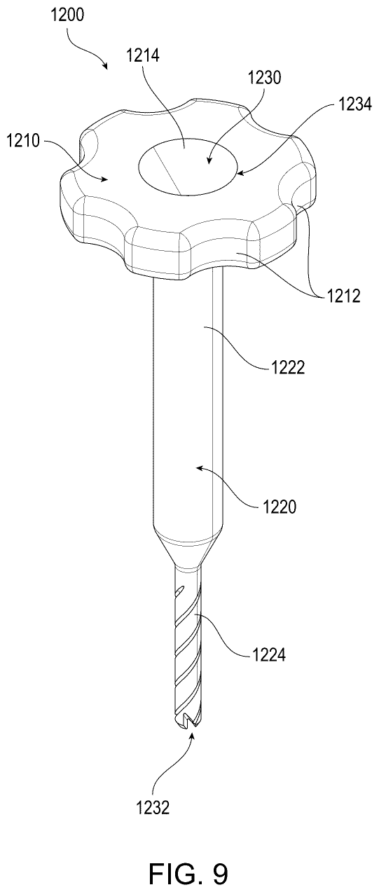

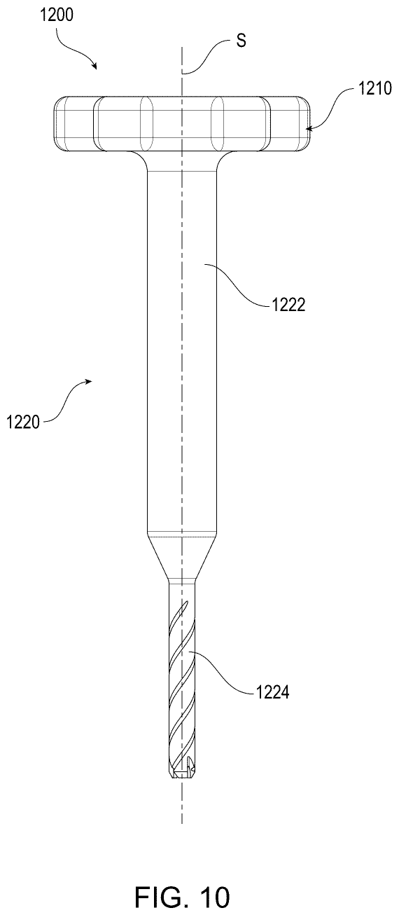

Referring to FIGS. 1, 5, 9 and 10, the system 1000 also includes a trocar 1200 configured to engage the guide instrument 1100 along the guide axis A. Although the guide axis A is shown as being centered within the second guide channel 1134b, the guide axis A may be selected such that the guide axis A is centered within the first guide channel 1134a or the third guide channel 1134c depending on the size of the anatomy of the patient. The trocar 1200 has a proximal head 1210, a shaft 1220 that extends from the proximal head 1210 in a distal direction along a shaft axis S, and a wire channel 1230 that extends from the proximal head 1210 through the shaft 1220 to a distal end of the trocar 1200. The proximal head 1210 includes one or more gripping members 1212. The shaft 1220 has a first portion 1222 that is sized and shaped to slidingly fit within one of the proximal guide channels 1134a, 1134b, 1134c, and a second portion 1224 configured to engage bone. The wire channel 1230 has a distal opening 1232 at the distal end, and a proximal opening 1234 at the proximal head 1210, where the proximal head 1210 defines a chamfer 1214 that extends to the proximal opening 1234 to facilitate insertion of the wire. The shaft 1220 of the trocar extends along the shaft axis S. M ore specifically, the first portion 1222 of the shaft 1220 has a first cross-sectional dimension that is perpendicular to the shaft axis S, the second portion 1224 of the shaft 1220 has a second cross-sectional dimension that is perpendicular to the shaft axis S and the second cross-sectional dimension is less than the first cross-sectional dimension. The second portion 1224 of the shaft 1220 includes cutting flutes or similar cutting surfaces for penetrating into and/or removing material from tissue and/or bone of a patient.

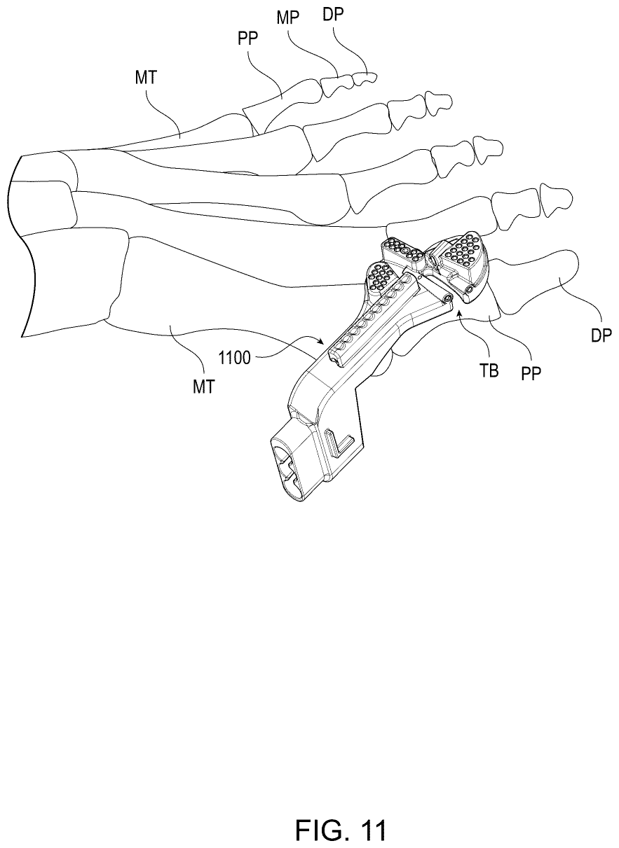

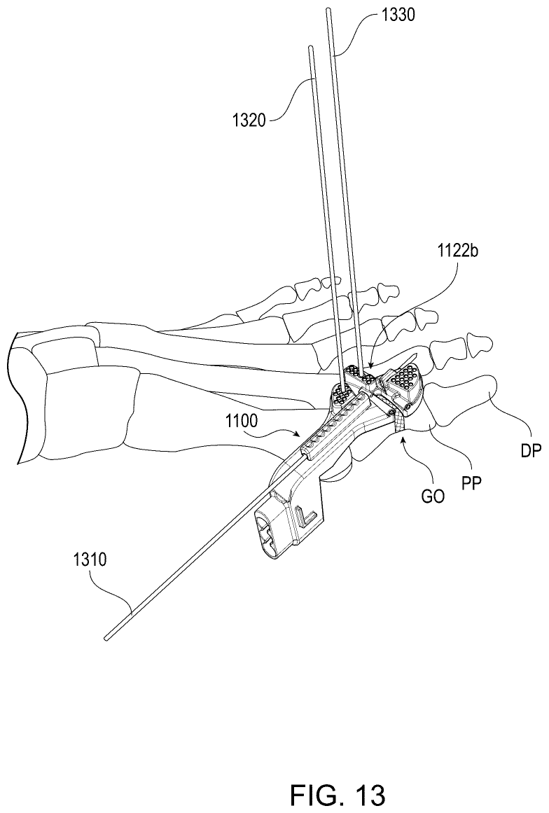

FIGS. 11-16 illustrate methods for using a guiding system 1000 as described herein. In use, as shown in FIGS. 11 and 12, the guide instrument 1100 is placed on the anatomy and the sighting wire 1310 is subsequently installed into the sighting channel 1142 of the sighting member 1140. Alternatively, the sighting wire 1310 may be installed into the sighting channel 1142 prior to the guide instrument 1100 being placed on the anatomy. For example, the guide instrument may be placed on or near a bone of the foot. Next, the user can visualize the guide instrument 1100 relative to the tissue or bone of the foot with reference to the radiopaque ring marker 1126. This allows the user to reposition the guide instrument 1100 into a preferred targeting direction. As shown in FIGS. 12, 13, and 14, once the guide instrument 1100 is in position, a proximal wire 1320 is inserted into a through-hole of the proximal grouping member 1122a of the guide instrument 1100. In some examples, the proximal wire 1320 may also be referred to as a first wire. The proximal wire 1320 is further inserted into a bone of the foot, such as but not limited to, a proximal phalanx PP, a middle phalanx M P, a distal phalanx DP, or metatarsal MT of the foot. In some embodiments, the proximal wire 1320 is inserted into a proximal phalanx PP of the large toe at a first location. Next, the central wire 1330 is inserted into a through-hole of the central grouping member 1122b. In some examples, the central wire 1330 may be referred to as a second wire. Once the proximal wire 1320 and the central wire 1330 are inserted and secured, the guide instrument 1100 is stabilized relative to the bone of the foot.

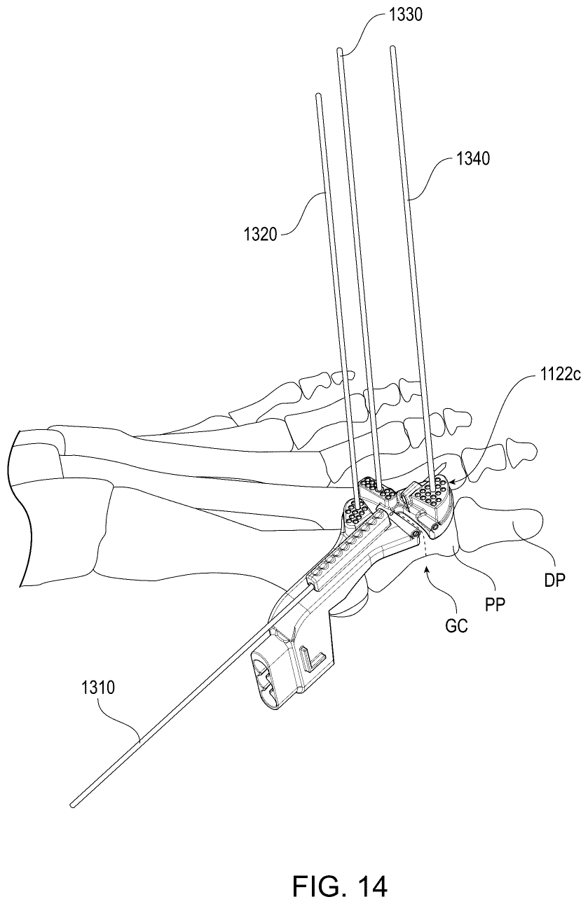

In FIG. 14, an osteotomy is created using the osteotomy window 1150 as a guide. The guide instrument 1100 is positioned by the user such that the target bone material TB is positioned generally between and below the opening of osteotomy window 1150 is removed. For example, a slice or wedge of the target bone material TB from the proximal phalanx PP is removed, creating an open gap GO between a proximal section of the proximal phalanx and a distal section of the proximal phalanx. The osteotomy window 1150 can be used by the user as landmarks, and in some embodiments, the user may elect to perform osteotomy outside of the opening of osteotomy window 1150. The osteotomy is closed manually by bringing the distal section of the bone (such as the distal end of the proximal phalanx) and the proximal section of the bone (such as the proximal end of the proximal phalanx) together to close the gap GC, and a distal wire 1340 is inserted in a through-hole of the distal grouping member 1122c to hold the distal section and the proximal section of the bon in place together. In some examples, the distal wire 1340 may be referred to as a third wire. The distal wire 1340 is further inserted into the bone of the foot. In some embodiments, the distal wire 1340 is inserted into the proximal phalanx PP of the large toe at a third location, the third location being distal relative to the second location.

The osteotomy is not shown and FIG. 14 reflects the procedure after the target bone material has been removed, the gap has been closed, and the distal wire 1340 has been inserted in the distal grouping member 1122c of the guide instrument 1100.

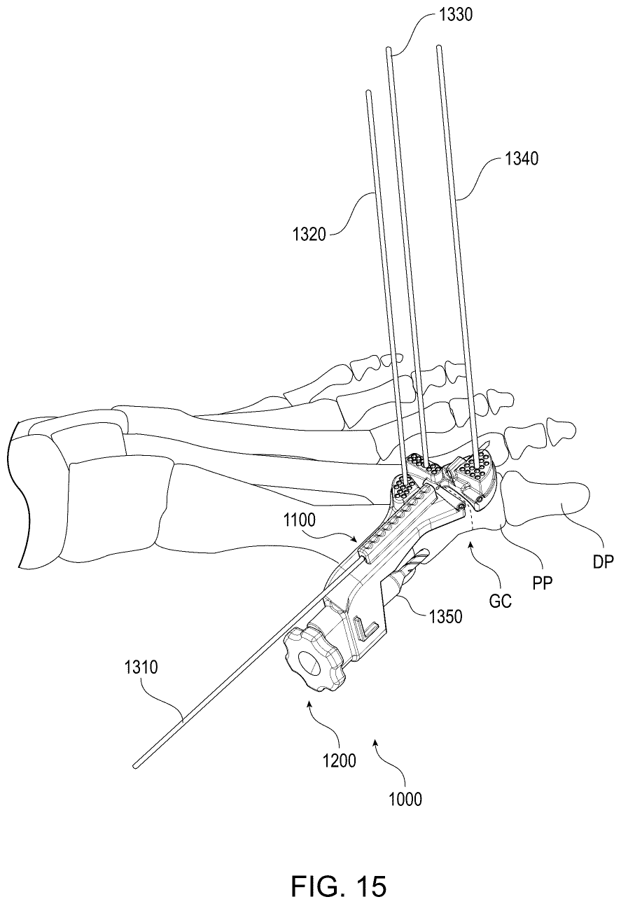

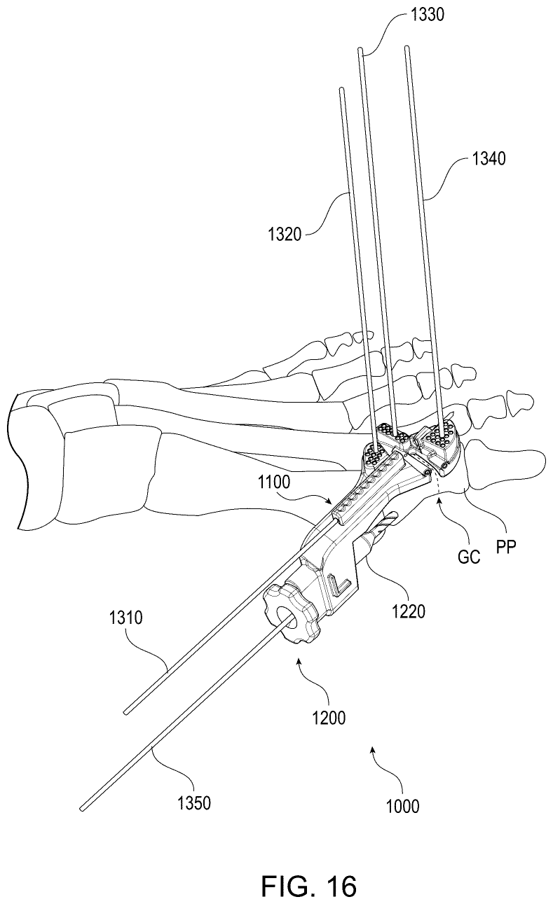

In FIG. 15, the trocar 1200 is inserted into the appropriate pre-set trajectory. More specifically, the shaft 1220 of the trocar 1200 is inserted into one of the guide channels 1134a, 1134b, 1134c of the guide barrels 1132a, 1132b, 1132c. In some embodiments, the distal end of the trocar 1200 is inserted into the bone by clockwise rotation and applying axial pressure inward. In FIG. 16, once the trocar 1200 has been inserted and secured to the bone, a screw k-wire 1350 is inserted into the trocar 1200. In some embodiments, the screw k-wire 1350 may be referred to as a fourth wire, and the screw k-wire 1350 may be inserted at a depth of about 3 to 5 mm into the bone. In some embodiments, the screw k-wire 1350 is inserted using a pecking technique and slow wire insertion to minimize skiving due to the oblique wire insertion angle. Alternatively, screw k-wire 1350 may be inserted into the bone first, and the trocar 1200 is subsequently placed over screw k-wire 1350 and then inserted into the bone. In some embodiments, the screw k-wire 1350 is further inserted into the bone at a desired depth using fluoroscopy.

Next the trocar 1200 is removed. In some embodiments, the trocar 1200 is removed from the bone by counter-clockwise rotation and axial pulling outward. In some embodiments, a pre-drill operation may be performed prior to the insertion of a cannulated screw, and the pre-drill operation may be beneficial for patients with bone that is harder or denser. For example, a 2.7 mm cannulated drill may be used to drill near the cortex to provide space for a 2.5 mm screw head of the cannulated screw. In some embodiments, a 1.9 mm cannulated drill may be used to drill a desire length to accommodate a length of the cannulated screw. After the trocar 1200 has been removed, and the pre-drill operation has been performed (if desired), a user then inserts a cannulated screw over the screw k-wire 1350, into the bone with the now closed gap f to locked the closed gap in place. The wires 1310, 1320, 1330, 1340, 1350 are removed and then guide instrument 1100 is removed from the anatomy.

Wherever possible, the same or like reference numbers are used throughout the drawings to refer to the same or like features. It should be noted that the drawings are in a simplified schematic form and are not drawn to precise scale. Certain terminology used in the description is for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the present disclosure in any manner not explicitly set forth. Additionally, the term “a”, as used in the specification, means “at least one.” The terminology includes the words above specifically mentioned, derivatives thereof, and words of similar import.

“Substantially” as used herein shall mean considerable in extent, largely but not wholly that which is specified, or an appropriate variation therefrom as is acceptable within the field of art. “Exemplary” as used herein shall mean serving as an example.

“About” as used herein when referring to a measurable value such as an amount, a temporal duration, and the like, is meant to encompass variations of ±20%, ±10%, ±5%, ±1%, or ±0.1% from the specified value, as such variations are appropriate.

Furthermore, the described features, advantages and characteristics of exemplary embodiments may be combined in any suitable manner in one or more embodiments. One skilled in the art will recognize, in light of the description herein, that the exemplary embodiments can be practiced without one or more of the specific features or advantages of a particular embodiment. In other instances, additional features and advantages may be recognized in certain embodiments that may not be present in all embodiments of the present disclosure.

While the disclosure is described herein, using a limited number of embodiments, these specific embodiments are not intended to limit the scope of the disclosure as otherwise described and claimed herein. The precise arrangement of various elements and order of the steps of articles and methods described herein are not to be considered limiting. For instance, although the steps of the methods are described with reference to a sequential series of reference signs and progression of the blocks in the figures, the method can be implemented in an order as desired.

1. A surgical system, comprising:
a guide instrument with a guide body having a proximal end, a distal end spaced from the proximal end along longitudinal axis, an upper surface and a lower surface spaced from the upper surface along a vertical axis that is perpendicular to a plane on which the longitudinal axis extends, the lower surface being configured to face tissue;
a distal platform having a set of spaced-apart grouping members along the upper surface, each grouping member having a plurality of through-holes that extend through the guide body, each through-hole configured to receive therethrough a separate fixation wire;
a sighting member carried by the distal platform, the sighting member defining a sighting channel that extends in direction parallel to or along the longitudinal axis; and
a back guide member located proximal with respect to the distal platform, the back guide member having a plurality of guide barrels each with a channel that extends along a direction aligned with or along the longitudinal axis.

2. The surgical system according to claim 1, further comprising a radiopaque ring carried by one of the grouping members, the radiopaque ring centered along a vertical axis that is perpendicular to the plane on which the longitudinal axis extends.

3. The surgical system according to claim 1, further comprising an osteotomy window extending into the distal platform along a direction that is transverse relative to the longitudinal axis and the vertical axis.

4. The surgical system according to claim 3, further comprising:
a first elongated radio-opaque element in the guide body adjacent to the osteotomy window; and
a second elongated radio-opaque element in the guide body adjacent to the osteotomy window and opposite the first elongated radio-opaque element.

5. The surgical system according to claim 3, wherein the osteotomy window defines an opening with a width of about 5 to 10 mm and a length of about 15 to 30 mm.

6. The surgical system according to claim 1, further comprising a trocar having a proximal head, a shaft that extends from the proximal head in a distal direction, a wire channel that extends from the proximal head through the shaft to a distal end of the trocar, the shaft having a first portion that is sized and shaped to slidingly fit within the one guide barrel of the plurality of guide barrels and a second portion configured to engage tissue or bone.

7. The surgical system according to claim 6, wherein the shaft of the trocar extends along a shaft axis, wherein the first portion of the shaft has a first cross-sectional dimension that is perpendicular to the shaft axis, and the second portion of the shaft has a second cross-sectional dimension that is perpendicular to the shaft axis, wherein the second cross-sectional dimension is less than the first cross-sectional dimension.

8. The surgical system according to claim 6, wherein the proximal head of the trocar includes one or more gripping members.

9. The surgical system according to claim 6, wherein the wire channel has a distal opening at the distal end, and a proximal opening at the proximal head, wherein the proximal head defines a chamfer that extends to the proximal opening to facilitate insertion of a screw k-wire.

10. The surgical system according to claim 6, wherein the second portion of the shaft includes cutting flutes.

11. The surgical system according to claim 1, wherein the back guide member extends from the lower surface of the guide body with a height of about 40 to 80 mm.

12. The surgical system according to claim 1, wherein a lower surface of the distal platform is a curved concave surface.

13. The surgical system according to claim 1, wherein the set of spaced-apart grouping members are each carried by a respective set of protrusions extending upwardly in a vertical direction from the upper surface of the guide body.

14. The surgical system according to claim 1, wherein:
the set of spaced-apart grouping members includes a proximal grouping member, a central grouping member, and a distal grouping member;
the proximal grouping member being proximal to the central grouping member relative to the longitudinal axis L; and
the distal grouping member being distal to the central grouping member relative to the longitudinal axis L.

15. The surgical system according to claim 14, wherein
the plurality of through-holes of the proximal grouping member consists of between about six and twelve through-holes;
the plurality of through-holes of the central grouping member consists of between about six and twelve through-holes; and
the plurality of through-holes of the distal grouping member consists of between about eight and eighteen through-holes.

16. A surgical system, comprising:
a surgical instrument with a guide body having a proximal end, a distal end spaced from the proximal end along longitudinal axis, an upper surface and a lower surface configured to face tissue;
a distal platform having a set of spaced-apart grouping members along the upper surface, each grouping member having a plurality of through-holes that extend through the guide body, each through-hole configured to receive therethrough a separate fixation wire;
a radiopaque ring carried by one of the grouping members, the radiopaque ring centered along a vertical axis that is perpendicular to a plane on which the longitudinal axis extends;
a sighting member carried by the distal platform, the sighting member defining a sighting channel that extends in direction parallel to or along the longitudinal axis;
a back guide member located proximal with respect to the distal platform, the back guide member having a plurality of guide barrels each with a channel that extends along a direction aligned with or along the longitudinal axis;
an osteotomy window extending into the distal platform along a direction that is transverse relative to the longitudinal axis and the vertical axis;
a first elongated radio-opaque element in the guide body adjacent to the osteotomy window;
a second elongated radio-opaque element in the guide body adjacent to the osteotomy window and opposite the first elongated radio-opaque element; and
a trocar having a proximal head, a shaft that extends from the proximal head in a distal direction, a wire channel that extends from the proximal head through the shaft to a distal end of the trocar, the shaft having a first portion that is sized and shaped to slidingly fit within the one of the guide barrels of the plurality of guide barrels and a second portion configured to engage tissue or bone.

17. A method, comprising:
placing a guide instrument on a tissue or bone of a foot;
visualizing a position of the guide instrument relative to the tissue or bone of the foot and adjusting the position of the guide instrument into a preferred targeting direction;
inserting a first wire in a first grouping member of the guide instrument until the first wire engages the bone of the foot;
inserting a second wire in a second grouping member of the guide instrument until the second wire engages the bone of the foot;
performing an osteotomy of a target bone of the foot by guiding a cutting instrument to a location between an osteotomy window that extends into a distal platform of the guide instrument;
reducing the osteotomy; and
inserting a third wire in a third group member of the guide instrument until the third wire engages the bone of the foot so that the reduced osteotomy is fixed in place.

18. The method of claim 17, further comprising:
inserting a trocar through a guide channel of a back guide member of the guide instrument until a distal end of the trocar engages the bone of the foot; and
inserting a fourth wire through a channel of the trocar.

19. The method of claim 18, further comprising:
removing the trocar while leaving the fourth wire in place; and
inserting a cannulated screw over the fourth wire and engaging the screw with the bone of the foot.

20. The method of claim 19, further comprising removing the fourth wire.
